(19) United States
(12) Patent Application Publication
(10) Pub. No.: US 2002/0061579 A1
(43) Pub. Date: May 23, 2002

(54) Counter selection strategy for Gram-negative bacteria

(76) Inventors: Stephen K. Farrand, Seymour, IL (US); Paul E. Staswick, Lincoln, NE (US); Thomas E. Clemente, Lincoln, NE (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/924,101

(22) Filed: Aug. 7, 2001

Related U.S. Application Data
(60) Provisional application No. 60/223,920, filed on Aug. 9, 2000.

(51) Int. Cl. A01H 1/00; C07H 21/04; C12N 1/21

(52) U.S. Cl. 435/252.2; 800/294; 536/23.2

(57) ABSTRACT

A Gram-negative bacterium useful for genetically engineering plants is provided. The Gram-negative bacterium contains, as part of genome, an inducible regulatory sequence operatively linked to a nucleotide sequence encoding a levansucrase. Alternatively, the Gram-negative bacterium comprises a recombinant nucleic acid construct containing an inducible regulatory sequence operatively linked to a nucleotide sequence encoding a levansucrase. Also provided are recombinant nucleic acid constructs comprising an inducible regulatory sequence operatively coupled to a nucleotide sequence encoding a levansucrase and a method for transforming plants using the Gram-negative bacterium of the present invention.

### BACKGROUND

Agrobacterium is a genus of soil Gram-negative bacteria that are widely used for the introduction of exogenous DNA into plants. The use of Agrobacterium species for DNA transfer is based on their natural ability to transfer DNA sequences into the genomes of plants. The most widely used species of Agrobacterium is A. tumefaciens the causal agent of the neoplastic disease crown gall in plants. A closely related species, A. rhizogenes, induces hairy root disease and also has been used for DNA transfer to plant genomes, but to a lesser extent. The ability of these bacteria to transfer DNA into plants depends on the presence of large plasmids (>100 kb) within the cells. These plasmids are referred to as the Ti (Tumor inducing) or Ri (Root inducing) in A. tumefaciens and A. rhizogenes, respectively. The mechanism for DNA transfer from the bacterium into the plant genome involves the mobilization of specific T-DNA (transfer DNA) molecules from the Ti plasmid into the host cell. The T-DNA region is delineated by 25 bp referred to as the left and right borders. In pathogenic Agrobacterium cells, within the T-DNA element reside genes for the over production of auxins and cytokinins which manifest the crown gall symptoms. The T-DNA element of pathogenic Agrobacterium strains also contains genes for the production of opines that are utilized by the bacterium as a nitrogen source.

Agrobacterium-mediated DNA transfer to plant cell genomes is usually conducted with “disarmed” (auxin, cytokinin and opine gene sequences removed from the T-DNA element) strains. In transformation studies, sequences of interest are introduced into the T-DNA region of a “disarmed” Agrobacterium strain. This chimeric T-DNA element can be carried on a separate, smaller, wide host range plasmid referred to as a binary vector or directly introduced into the resident “disarmed” Ti plasmid. The first step in the basic Agrobacterium-mediated transformation protocol, requires the inoculation of plant cells with transconjugants of “disarmed” Agrobacterium cells carrying the sequences of interest on the chimeric T-DNA element. The plant cells are subsequently cultured for a period generally ranging from one to seven days in a step of the protocol referred to as co-cultivation. Following the co-cultivation period, the plant cells are subcultured on regeneration medium for whole plant development.

A critical step in this process is the elimination of the Agrobacterium cells during plant development. Currently, Agrobacterium-mediated DNA transfer protocols incorporate antibiotics into the regeneration medium as a strategy to counter select Agrobacterium cells. Although successful, this approach adds significant cost to the transformation process. Moreover, there have been reports demonstrating a negative impact on plant tissue using medium supplemented with the antibiotics commonly used to eliminate Agrobacterium cells such as ticarcillin, cefotaxime, carbenicillin or vancomycin.

The saeB gene from Bacillus subtilis encodes for the enzyme levansucrase, which hydrolyzes sucrose to produce the polysaccharide levan, the presence of which causes the lysis of several Gram-negative bacteria, and in particular Agrobacterium (Gay et al. (1985)) J. Bacteriol. 164:918-921). Expression of sacB is controlled by its regulatory sequence sacR. The present inventors have discovered that the introduction of a sequence encoding a levansucrase and in particular the sacB open reading frame (ORF), under strict control of an inducible regulatory sequence allows for the selection of Agrobacterium cells without the use of antibiotics. Thus, the present invention overcomes the problems of cost and negative effects on plant culture associated with the use of antibiotics for counter selection of the Agrobacterium cells.

### SUMMARY

Among the several aspects of the invention is provided a Gram-negative bacterium comprising an inducible regulatory sequence operatively linked to a nucleotide sequence encoding a levansucrase contained within the genome of said Gram-negative bacterium.

Another aspect provides, a Gram-negative bacterium comprising a recombinant nucleotide sequence containing an inducible regulatory sequence other than sacR, operatively linked to a nucleotide sequence encoding a levansucrase.

Yet another aspect provides, a recombinant nucleic acid construct comprising an inducible regulatory sequence other than sacR, operatively linked to a nucleotide sequence encoding a levansucrase.

Still another aspect provides, a method for transforming a plant cell comprising obtaining an Agrobacterium strain whose genome contains an inducible regulatory sequence operatively linked to a nucleotide sequence encoding a levansucrase; introducing a DNA construct into a T-DNA element of the Agrobacterium; and inoculating at least one plant cell with the Agrobacterium containing the construct for a time sufficient for mobilization of the T-DNA element from the Agrobacterium to the plant genome.

Another aspect provides, a method for transforming a plant cell comprising obtaining an Agrobacterium strain comprising a first recombinant nucleic acid construct containing an inducible regulatory sequence other than sacR, operatively linked to a nucleotide sequence encoding a levansucrase; introducing a second DNA construct into a T-DNA element of the Agrobacterium; and inoculating at least one plant cell with the Agrobacterium containing the first and second constructs for a time sufficient for mobilization of the T-DNA element from the Agrobacterium to the plant genome.

A further aspect provides, a method for counter selecting against a Gram-negative bacterium whose genome contains an inducible regulatory sequence operatively linked to a nucleotide sequence encoding a levansucrase comprising, introducing, in the presence of sucrose, a suitable inducer to cause the production of levansucrase by the bacterium resulting in the lysis of the bacterium.

Still a further aspect provides, a method for counter selecting against a Gram-negative bacterium containing a recombinant nucleic acid construct that includes an inducible regulatory sequence other than sacR, operatively linked to a nucleotide sequence encoding a levansucrase, comprising introducing, in the presence of sucrose, a suitable inducer to cause the production of levansucrase by the bacterium resulting in lysis of said bacterium.

Another aspect provides, a vector comprising a recombinant nucleic acid construct containing an inducible regulatory sequence other than sacR, operatively linked to a nucleotide sequence encoding a levansucrase.

### DEFINITIONS

As used herein, “regulatory sequence” means a sequence of DNA concerned with controlling expression of a gene; e.g. promoters, operators and attenuators. A regulatory sequence, may, potentially operate in conjunction with the biosynthetic apparatus of a cell.

As used herein, “polynucleotide” and “oligonucleotide” are used interchangeably and mean a polymer of at least two nucleotides joined together by a phosphodiester bond and may consist of either ribonucleotides or deoxynucleotides.

As used herein, “sequence” means the linear order in which monomers in a polymer, for example, the order of amino acids in a polypeptide or the order of nucleotides in a polynucleotide.

As used herein, “peptide”, and “protein” are used interchangeably and mean a compound that consist of two or more amino acids that are linked by means of peptide bonds.

As used herein, “levansucrase” means a protein, a protein fragment or peptide that has the property of synthesizing a carbohydrate polymer consisting of repeating fructose residues, using sucrose as a substrate. The repeating fructose residues may be linked by β-1 linakge or a β-2-6 linkage or any combination of the two linkage types. The polymer of repeating fructose units may contain one terminal glucose residue, derived from a sucrose molecule, and at least two fructose residues.

As used herein, “inducer” means a substance that interacts with a regulatory sequence, either directly or indirectly, to increase the rate of transcription of the nucleotide sequence controlled by the regulatory sequence.

LB means 10 g tryptone, 5 g yeast extract, 5 g NaCl and 1 ml 1N NaOH per liter H₂O.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the inventive discovery.

All publications, patents, patent applications and other references cited in this application are herein incorporated by reference in their entirety as if each publication, patent, patent application or other reference were specifically and individually indicated to be incorporated by reference.

The present invention provides a method for transforming plant cells and constructs and bacteria useful in said method. The invention involves the insertion of a sequence encoding a levansucrase, and in particular the sacB gene ORF, under strict control of an inducible regulatory sequence into a Gram-negative bacterium and in particular Agrobacterium tumefaciens. The sacB gene encodes levansucrase (sucrose 2,6,β-D-fructan 6-β-D-glucosyltransferase; (IC 2.3.1.10)), a 50 kD enzyme secreted by B. subtilis after induction by sucrose. Levansucrase catalyzes transfructorylation from sucrose to various acceptors. The two main physiological reactions resulting are, 1) levan synthesis and 2) sucrose hydrolysis. Since sucrose is the primary carbon source used in most plant tissue culture medium formulations, the present invention allows for the efficient counter selection of the bacterium without the use of antibiotic supplements.

One aspect provides a Gram-negative bacterium useful for the transfer of heterologous polynucleotide sequences into a host cell. The bacterium has as part of its genome a recombinant nucleic acid sequence comprising an inducible regulatory sequence operatively linked to a nucleotide sequence encoding the enzyme levansucrase. Alternatively, the bacterium comprises a recombinant nucleic acid sequence comprising an inducible regulatory sequence other than sacR, operatively linked to a nucleotide sequence encoding the enzyme levansucrase. A nucleic acid sequence is operatively linked when it is placed into a functional relationship with another nucleic acid sequence. For example, DNA for a presequence or secretory leader is operatively linked to a DNA element encoding for a polypeptide to be expressed as a preprotein which participates in the secretion of the polypeptide; a promoter or regulatory sequence is operatively linked to a coding sequence if it affects the transcription of the coding sequence; or a ribosome binding site is operatively linked to a coding sequence if it is positioned so as to facilitate translation. Any nucleic acid sequence encoding a protein, polypeptide or protein fragment that is functional as a levansucrase can be used. Numerous sequences encoding levansucrase are known in the art and can be found in publicly available databases such as those maintained by the National Center for Biotechnology Information and available at http://www.nobi.nlm.nih.gov. Representative examples include, without limitation, sequences encoding levansucrases obtained from Acetobacter xylinus (GenBank AB034152), Gluconacetobacter diazotrophicus (GenBank L41732), Zymomonas mobilis (GenBank L33402), Paenibacillus polymyza (GenBank AJ133737), Rahnella aquatillis (GenBank U91484), Pseudomonas syringae (GenBank AF052289), Bacillus stearothermophilus (GenBank U34874), Bacillus subtilis (GenBank X02730) and Bacillus amyoliquefaciens (GenBank X52988). Copies of the GenBank records for the listed accession numbers are attached and are to be considered part of this application. In one embodiment, the polynucleotide sequence encoding levansucrase enzyme is the coding region of the sacB gene of Bacillus subtilis. As used herein, the term “coding region” refers to the nucleotide sequence of a gene that is translatable into a polypeptide. Methods for producing artificial nucleotide sequences such as by cloning or nucleotide synthesis are well known in the art. Such sequences are included within the scope of the invention as long as they encode a biological equivalent to a levansucrase.

The regulatory sequences can consist of an inducible promoter, in combination with an operator sequence. As used herein, the term “operator” or “operator sequence” refers to a polynucleotide sequence to which a repressor protein can bind, thereby regulating the expression of a gene. Any inducible promoter that is functional within Gram-negative bacterium can be used. It is preferred that the promoter or combination of the promoter and operator be strictly inducible so that there is little or no production of levansucrase in the absence of the inducing agent. In one preferred embodiment, the regulatory sequence is one that is functional in members of the genus Agrobacterium, and in particular A. tumefaciens. Examples of suitable regulatory sequences include, but are not limited to, the Plac promoter and operator of E. coli, the nocR gene, which encodes for the transcriptional activator of Pi2 (noc), and in the presence of the noc1 operon which encodes for the nopaline transport system of A. tumefaciens (Von Lintig et al. (1991) Molec. Plant Microbe Interaction, 4:370-378) and the P_{BAD} promoter and araC operator of E. coli (Gallegos et al. (1997) Microbiol. Mol. Biol. Rev. 61:393-410).

Another aspect provides a recombinant nucleic acid construct comprising an inducible regulatory sequence operatively linked to a nucleotide sequence encoding a levansucrase. Any previously mentioned regulatory sequence or levansucrase encoding sequences can be used, although other suitable sequences will be apparent to those of ordinary skill in the art and are considered within the scope of the present invention. As used herein, “recombinant construct” is defined either by its method of production or its structure. In reference to its method of production, e.g., a product made by a process, the process is use of recombinant nucleic acid techniques involving human intervention in the nucleotide sequence. Alternatively, in terms of structure, it can be a sequence comprising fusion of two or more nucleic acid sequences which are not naturally contiguous or operatively linked to each other.

The recombinant constructs of the present invention are produced using methods well known to those of ordinary skill in the art which can be found, for example, in standard texts such as Sambrook et al. Molecular Cloning, 2^{nd} ed., Cold Spring Harbor Laboratory Press, 1989 and Ausubel, et al. Short Protocols in Molecular Biology, 3^{rd} ed., Wiley & Sons, 1995. In general, recombinant constructs are produced by a series of restriction enzyme digestions and ligation reactions which result in the sequences being assembled in the desired configuration. If suitable restriction sites are not available, alternative strategies, for example, the use of synthetic oligonucleotide linkers and adaptors, which are well known to those skilled in the art and described in the references cited above, can be employed to assemble the desired recombinant constructs.

It will be apparent to those of ordinary skill in the art, that the precise restriction enzymes, linkers and/or adaptors required as well as the precise reaction conditions will vary with the sequences and cloning strategies used. The assembly of recombinant constructs, however, is routine in the art and can be readily accomplished by the skilled technician without undue experimentation. Non-limiting illustrations of the assembly of recombinant constructs useful in the present invention can be found in the examples that follow.

Once made, the recombinant constructs can be inserted into the genome of Gram-negative bacterium or introduced separately on a self replicating plasmid of a Gram-negative bacterium used in transforming host cells. In one embodiment, the Gram-negative bacterium is Agrobacterium tumefaciens. The inventors have found that insertion of the recombinant construct into the genome of the bacterial vector is superior to the method of placing the construct in A. tumefaciens via a self replicating plasmid. As shown in Example 1, it was discovered that a self replicating plasmid carrying the sacB coding region was frequently lost resulting in overgrowth of the bacterial cells during plant regeneration.

Any method capable of introducing the construct into the genome of the bacterial vector can be used. In one embodiment, the construct is inserted by the use of homologous recombination in particular the method of Ruvkun and Ausubel ((1981) Nature, 289:85-88). In this method, a mutation, in the form of the recombinant construct of the present invention, is directed to a specific locus on the chromosome by homologous exchange recombination. Any locus which allows the inducible expression of levansucrase and does not impede with the DNA transfer machinery can be used. In one embodiment, the construct is inserted at the tetR/tetA loci of Agrobacterium (Luo and Farrand (1999) J. Bacteriol 181:618-626).

Another aspect provides method for transforming a plant cell using an Agrobacterium tumefaciens bacterium of the present invention as a vector. In general the method involves obtaining an A. tumefaciens strain whose genome includes a nucleotide sequence encoding a levansucrase operatively linked to an inducible regulatory sequence as described above or an A. tumefaciens strain that contains a recombinant nucleotide sequence encoding a levansucrase operatively linked to an inducible regulatory sequence as described above. The nucleotide sequence(s) of interest that are to be transferred to the plant cell can be inserted within the T-DNA element and introduced either directly to the resident Ti plasmid or separately using a binary plasmid strategy. Methods for the introduction of exogenous nucleotide sequences into the T-DNA element and the use of the derived Agrobacterium transconjugant to transform plant cells are well known in the art (see, Maliga et al. Methods in Plant Molecular Biology, Cold Spring Harbor Laboratory Press, 1995). Once the sequence to be delivered to the plant cell is assembled into the T-DNA element and introduced either directly to the resident Ti plasmid or via the binary vector strategy to the Agrobacterium of the present invention, the bacterium is subsequently used to inoculate plant cells either by direct injection or by co-cultivating the bacterium of the present invention with individual plant cells or pieces of plants such as leaf discs. Co-cultivation is carried out in medium supplemented with a carbon source, preferably glucose, for a sufficient amount of time to allow the T-DNA element to be mobilized from the bacterium to the plant cell genome. The co-cultivation period is determined empirically, but generally ranges from one to seven days. Co-cultivation periods may vary for a particular plant species, but determinations are routine in the art and can be made by one of ordinary skill in the art without undue experimentation. Following co-cultivation, the transforming bacteria are counter selected prior to the regeneration of the plant cells to whole plants. Typically, the transforming bacteria are removed by using antibiotic supplements to the regeneration medium. In the present invention, however, an inducing agent that activates the promoter linked to the levansucrase coding region is added to sucrose amended regeneration medium. Activation of the inducible promoter results in the production of levan which causes the lysis of the Agrobacterium cells, thus providing efficient counter selection strategy. As a result, the regeneration of whole plants from the inoculated plant cells can be carried out, in the absence of antibiotics typically used to counter select Agrobacterium cells, following standard protocols (see, Maliga et al. supra).

An alternative embodiment provides an Agrobacterium tumefaciens vector in which the nucleotide sequence encoding a levansucrase operatively linked to an inducible regulatory sequence is contained in the Agrobacterium as part of a binary vector system. Binary vector systems and their constructions are well known in the art and are described, for example, in Maliga et al. supra and The Encyclopedia of Molecular Biology, J. Kendrew, ed., Blackwell Science, 1994). The resultant Agrobacterium strain can subsequently be used to genetically engineer plant cells as described above.

### EXAMPLES

### Example 1

### Counter Selection Using sacB in a Binary Vector System

The npt1-sacB-sacR loci from the vector pUM24 (Reid and Collmer (1987) Gene 57:239-246) was subcloned as a BamHI fragment into the binary vector pZP112 (Hajdukiewicz et al. (1994) Plant Molec. Biol. 25:989-994), at the BclI site just outside the left border region. The resultant counter selection (suicide) vector was referred to as pPTN114. The binary vector was mobilized into A. tumefaciens strains C58C1 (Koncz and Schell (1986) Mol. Gen Genet. 204:383-396) and EHA105 (Hood et al (1993) Transgenic Research 2:208-218) via tri-parental mating (Ditta et al (1980) Proc. Natl. Acad. Sci USA 77:7347-7351). The C58C1 transconjugant was used for subsequent evaluation in tobacco transformations, while the EHA105 transconjugant was tested in soybean transformations.

Culturing of the C58C1 and EHA105 carrying the binary vector pPTN114 on LB medium supplemented with 1% to 3% sucrose was lethal, while LB medium supplemented with 1.5% glucose was conducive to cell growth. Tobacco and soybean transformations were initiated to evaluate the efficacy of incorporating the sacB locus in a binary vector as a counter selection strategy for A. tumefaciens in the absence of antibiotics. In both plant species, Agrobacterium growth was impeded for approximately 10 days, at which point bacterial growth was observed on the plant tissue culture medium containing 3% sucrose. The bacteria from the tissue culture failed to grow when transferred onto LB medium supplemented with kanamycin. These results suggested that the Agrobacterium cells had lost the binary vector.

### Example 2

### Counter Selection by Genomic Incorporation of sacB

A construct was assembled that specifically targeted the sacB locus to the tetR/tetA loci in A. tumefaciens. An EcoRI fragment from pSWE8.5 bearing the tetR/tetA loci was subcloned into pGEM T-Easy. The resulting plasmid was digested with Hind III and a 0.5 kb fragment at the tetR locus was replaced with the 3.8 kb BamHI insert from pUM24 containing the nptI-sacB-sacR region (Reid and Collmer (1987) Gene 57:239-246). This step was accomplished by annealing after adding homopolymeric G and C tails to the vector and insert, respectively. The resulting construct was electroporated into Agrobacterium strain NT1/pEHA105 and transformants were selected on kanamycin (50 mg/L) LB plates. Since the pGEM backbone was not expected to replicate in A. tumefaciens, kanamycin resistant transformants were presumed to be due to chromosomal integration of the nptI-sacB-sacR cassette at the tetR/tetA loci. Individual colonies were picked and replica plated to kanamycin supplemented LB medium with and without sucrose (3% w/v). The resulting bacterial patches that showed little or no growth on sucrose, but vigorous growth on kanamycin alone, were recovered, diluted and spread on kanamycin plates to isolate individual colonies. These were again tested as before until an isolate that consistently gave no growth on sucrose was recovered.

### Example 3

### Use of the E. coli Plac/repressor System to Control sacB Expression

The E. coli lactose operon is tightly regulated by the presence of a 21 bp operator that resides immediately down stream of the Plac promoter. In the absence of β-galactose sugar, the lac repressor will bind to the cis operator and prevent RNA polymerase initiation. In the presence of a β-galactose sugar, the lac repressor cannot bind to the operator and thus RNA polymerization proceeds. This system may be exploited as a strategy for tight regulation of the sacB expression in Agrobacterium tumefaciens cells.

The sacB open reading frame (ORF) can be subcloned downstream of the Plac promoter element coupled with the 21 bp operator sequence. The lacI cassette coding for the lac repressor may be ligated to the derived sacB cassette. The genetic element carrying the Plac-sacB and lac repressor cassettes can be introduced to the chromosome of Agrobacterium tumefaciens via homologous recombination. The preferred site for the recombination event would be the tetR/tetA loci of Agrobacterium recently described by Luo and Farrand ((1999) J. Bacteriol. 181:618-626).

This strategy will be useful for the genetic engineering of both monocotyledonous and dicotyledonous plant species. Various steps are followed in the Agrobacterium-mediated transformation of plant species. Generally the first step involves the inoculation of the explant (plant cells or tissue segments) with Agrobacterium tumefaciens cells. The explant can be leaf segment, cotyledon, stem, root, flower part or cells thereof. After a period of one to seven days, generally termed the co-cultivation period, the explant is transferred to plant regeneration medium supplemented with sucrose as the carbon source. The sacB system in this example may be induced by the addition of isopropyl-β-D-thiogalactoside (IPTG) at levels ranging from 0.1 μM up to 1 mM and/or lactose at levels from 0.1 μM to 1 mM.

### Example 4

### Use of the Nopaline—Inducible Marker System to Regulate sacB Expression

The Pi2(noc) promoter (Von Lintig et al. (1991) Molec. Plant Microbe Interaction 4:370-378) from Agrobacterium tumefaciens is induced in the presence of nopaline. This regulatory sequence may be employed as a strategy to regulate sacB expression in Agrobacterium tumefaciens cells. In this example, the sacB ORF will be subcloned down stream of the Pi 2(noc) promoter. The resultant cassettes preferably will be introduced into the chromosome of Agrobacterium tumefaciens via homologous recombination. The preferred site for recombination is the tetR/tetA loci. The nocR gene, which encodes for the transcriptional activator of pi2(noc), and the nod operon which encodes for the nopaline transport system can be supplied by the vir region of the resident Ti plasmid, or by cloning these two loci onto a self-replicating plasmid. In this example the sacB counter selection (suicide) system may be induced upon the addition of nopaline at levels ranging from 50 μg/L up to 200 μg/L.

### Example 5

### Use of the E. coli araC Regulator to Control sacB Expression

The E. coli P_{BAD} promoter is highly induced in the presence of L-arabinose. The induction of the system is controlled by the presence of a cis acting element upstream of the P_{BAD} promoter, araC (Gallegos et al. (1997) Microbiol. Molec. Biol. Rev. 61:393-410). This system can be utilized to regulate the expression of the sacB in Agrobacterium tumefaciens cells by placing the araC cis element just 5′ to the P_{BAD} promoter (Luo and Farrand (1999) J. Bacteriol. 181:618-626); Newman and Fuqua (1999) Gene 227:197-203) and subsequently subcloning the sacB open reading frame downstream of the assembled DNA elements. In this example, counter selection of the bacterial cells can be induced following the co-cultivation period in plant transformation protocols by supplementing the regeneration medium with levels of L-arabinose ranging from 5 g/L up to 20 g/L.

### Example 6

### Use of the traCDG Promoter to Control sacB Expression

The sacB gene can be placed under the control of the traCDG promoter (Farrand et al., (1996) Bacteriol. 178:4233-4247; Oger et al., (1998) Mol. Microbiol. 27:277-288; Luo and Farrand, (1999) Proc. Natl. Acad. Sci. USA 96:9009-9014). Initiation of this promoter is absolutely dependent upon activated TraR. Second, the TraR will be placed under the direct control of an opine-responsive promoter system. We will fuse traR directly to a fragment of DNA containing the occ promoter from the octopine-type Ti plasmid pTiR10. This promoter is activated by OccR, a lysR-like activator in response to the opine, octopine (Habeeb, et al., 1991). The occR gene is located directly adjacent to the occ promoter and will be included in the recombinant construct. One can provide a copy of traM, which encodes for the antiactivator, in the traR-pOcc-occ construct. When the two constructs are combined in an Agrobacterium host, expression of sacB should be strongly suppressed in the absence of the opine. However, addition of octopine (which will activate expression of traR leading to accumulation of the activator to levels that overcome the antiactivator, TraM) should strongly induce sacB.

The genetic elements described above can be introduced to a neutral site in the chromosome of Agrobacterium tumefaciens, with respect to plant transformation effects, via homologous recombination. The preferred site for the recombination event would be the tetR/tetA loci of Agrobacterium. However, with the imminent availability of the genome sequence of Agrobacterium strain C58 one should be able to identify alternative sites within the chromosome.

### Example 7

### Use of a Second Copy of the sacB Gene

To circumvent potential mutational inactivation of sacB a second copy of the gene can be provided. To prevent recombination between the two copies one can use a sacB gene from another bacterium. Possibilities of alternative sacB sources include B. stearothermophilus, B. amyloliquefaciens, or Streptococcus mutans. The sequence for each of these is available in the data bases. The alternative sacB gene can be fused to a second copy of the TraR-dependent traCDG promoter.

### CONCLUSION

In light of the detailed description of the invention and the examples presented above, it can be appreciated that several aspects of the invention are achieved.

It is to be understood that the present invention has been described in detail by way of illustration and example in order to acquaint others skilled in the art with the invention, its principles, and practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A Gram-negative bacterium comprising, an inducible regulatory sequence operatively linked to a nucleotide sequence encoding a levansucrase contained within the genome of said Gram-negative bacterium.

2. A Gram-negative bacterium comprising a recombinant nucleotide sequence containing an inducible regulatory sequence other than sacR operatively linked to a nucleotide sequence encoding a levansucrase.

3. The Gram-negative bacterium of claim 1, wherein said nucleotide sequence encoding a levansucrase is a sacB open reading frame.

4. The Gram-negative bacterium of claim 1, wherein said bacterium is a member of the genus Agrobacterium

5. The Gram-negative bacterium of claim 3, wherein said bacterium is Agrobacterium tumefaciens.

6. The Gram-negative bacterium of claim 1, wherein said regulatory sequence comprises the E. coli lactose operon.

7. The Gram-negative bacterium of claim 1, wherein said regulatory sequence comprises the Pi 2(noc) promoter and the noc 1 operon.

8. The Gram-negative bacterium of claim 1, wherein said regulatory sequence comprises the P_{BAD} promoter and araC cis element.

9. A recombinant nucleic acid construct comprising an inducible regulatory sequence other than sacR, operatively linked to a nucleotide sequence encoding a levansucrase.

10. The recombinant nucleic acid construct of claim 9, wherein said regulatory sequence comprises the E. coli lactose operon.

11. The recombinant nucleic acid construct of claim 9, wherein said regulatory sequence comprises the Pi2 (noc) promoter and the noc 1 operon.

12. The recombinant nucleic acid construct of claim 9, wherein said regulatory sequence comprises the P_{BAD} promoter and the araC cis element.

13. The recombinant nucleic acid construct of claim 9, wherein said sequence encoding a levansucrase is a sacB open reading frame.

14. A method for transforming a plant cell comprising,
a) obtaining an Agrobacterium whose genome contains an inducible regulatory sequence operatively linked to a nucleotide sequence encoding a levansucrase;
b) introducing a DNA construct into a T-DNA element of said Agrobacterium; and
c) inoculating at least one plant cell with the Agrobacterium of (b) for a time sufficient for mobilization of the T-DNA element from the Agrobacterium to the plant genome.

15. A method for transforming a plant cell comprising,
a) obtaining an Agrobacterium comprising a first recombinant nucleic acid construct containing an inducible regulatory sequence other than sacR, operatively linked to a nucleotide sequence encoding a levansucrase;
b) introducing a second DNA construct into a T-DNA element of said Agrobacterium; and
c) inoculating at least one plant cell with the Agrobacterium of (b) for a time sufficient for mobilization of the T-DNA element from the Agrobacterium to the plant genome.

16. The method of claim 14, further comprising counter selecting against said bacterium by introducing, in the presence of sucrose, a suitable inducer to cause the production of levansucrase by the bacterium resulting in the lysis of said bacterium.

17. The method of claim 14, wherein said regulatory sequence comprises the E. coli lactose operon.

18. The method of claim 14, wherein said regulatory sequence comprises the pi 2(noc) promoter and noc 1 operon.

19. The method of claim 14, wherein said regulatory sequence comprises the P_{BAD} promoter and the araC cis element.

20. The method of claim 14, wherein said sequence encoding a levansucrase is a sacB open reading frame.

21. A method for counter selecting against a Grain-negative bacterium whose genome contains an inducible regulatory sequence operatively linked to a nucleotide sequence encoding a levansucrase comprising, introducing, in the presence of sucrose, a suitable inducer to cause the production the levansucrase by the bacterium resulting in the lysis of said bacterium.

22. A method for counter selecting against a Gram-negative bacterium containing a recombinant nucleic acid construct that includes an inducible regulatory sequence other than sacR, operatively linked to a nucleotide sequence encoding a levansucrase comprising, introducing, in the presence of sucrose, a suitable inducer to cause the production of levansucrase by the bacterium resulting in the lysis of said bacterium.

23. The method of claim 21, wherein said bacterium is a member of the genus Agrobacterium

24. The method of claim 23, wherein said bacterium is an Agrobacterium tumefaciens bacterium.

25. The method of claim 21, wherein said regulatory sequence comprises the E. coli lactose operon.

26. The method of claim 21, wherein said regulatory sequence comprises the Pi 2(noc) promoter and noc 1 operon.

27. The method of claim 21, wherein said regulatory sequence comprises the P_{BAD} promoter and the araC cis element.

28. The method of claim 21, wherein said sequence encoding a levansucrase is a sacB open reading frame.

29. A vector comprising a recombinant nucleic acid construct containing an inducible regulatory sequence other than sacR, operatively linked to a nucleotide sequence encoding a levansucrase.

30. The vector of claim 29, wherein said regulatory sequence comprises the E. coli lactose operon.

31. The vector of claim 29, wherein said regulatory sequences comprises the Pi 2(noc) promoter and noc 1 operon.

32. The vector of claim 29, wherein said regulatory sequences comprises the P_{BAD} promoter and the araC cis element.

33. The vector of claim 29, wherein said sequences encoding a levansucrase is a sacB open reading frame.

34. The Gram-negative bacterium of claim 1, wherein the regulatory sequence comprises the traCDG promoter and the occ promoter.

35. The Gram-negative bacterium of claim 3, wherein the nucleotide sequence encoding a levansucrase contains a second copy of a sacB open reading frame.

36. The recombinant nucleic acid construct of claim 9, wherein the regulatory sequence comprises the traCDG promoter and the occ promoter.

37. The recombinant nucleic acid construct of claim 13, wherein the nucleotide sequence encoding a levansucrase contains a second copy of a sacB open reading frame.

38. The method of claim 14, wherein the regulatory sequence comprises the traCDG promoter and the occ promoter.

39. The method of claim 20, wherein the nucleotide sequence encoding a levansucrase contains a second copy of a sacB open reading frame.

40. The method of claim 21, wherein the regulatory sequence comprises the traCDG promoter and the occ promoter.

41. The method of claim 28, wherein the nucleotide sequence encoding a levansucrase contains a second copy of a sacB open reading frame.

42. The vector of claim 29, wherein the regulatory sequence comprises the traCDG promoter and the occ promoter.

43. The vector of claim 33, wherein the nucleotide sequence encoding a levansucrase contains a second copy of a sacB open reading frame.
